(19) United States
(12) Patent Application Publication
(10) Pub. No.: US 2014/0288495 A1
(43) Pub. Date: Sep. 25, 2014

(54) Timed release capsule and device for use with beverages

(71) Applicant: Alejandro I. Olmos, Chula Vista, CA (US)

(72) Inventor: Alejandro I. Olmos, Chula Vista, CA (US)

(21) Appl. No.: 14/296,392

(22) Filed: Jun. 4, 2014

Related U.S. Application Data
(63) Continuation-in-part of application No. 13/570,066, filed on Aug. 8, 2012.
(60) Provisional application No. 61/521,978, filed on Aug. 10, 2011.

(51) Int. Cl. A23L 2/56; A61J 7/00; A61K 9/48; A47G 21/18

(52) U.S. Cl. CPC A23L 2/56; A47G 21/183; A61J 7/0038; A61K 9/4891
USPC 604/77; 426/89; 426/85; 424/457

(57) ABSTRACT

Timed release capsules administered in beverage preparations may be used for delayed release of flavor or color enhancements, and pharmaceuticals. A composite timed release capsule includes: a first layer, including a first material; a second layer, including a second material, such that the second material further includes inner bodies; and an inner layer, which includes a third material. A composite straw includes: a standard straw; a snap-off cap; an upper stop; a lower stop; timed release capsules, retained in position between the snap-off cap and the upper stop; composite timed release capsules, retained in position between the upper stop and the lower stop. A user can remove the snap-off cap to pour the timed release capsules into a drinking container, insert the composite straw into the drinking container, where the composite timed release capsules dissolve, and embedded inner bodies fall into the beverage.

### CROSS-REFERENCE TO RELATED APPLICATIONS

This application is a continuation-in-part of U.S. application Ser. No. 13/570,066, filed Aug. 8, 2012, which is the non-provisional application for U.S. Provisional Application No. 61/521,978 filed Aug. 10, 2011.

### FIELD OF THE INVENTION

The present invention relates generally to the field of timed-release capsules administered in beverage preparations.

### INCORPORATION BY REFERENCE

The following patent applications have been incorporated by reference: US patent 2010/0136183 by Gonus et al., US patent application 2009/0291121 by HAAS et al., US patent application 207/0071808 by Janik et al., US application 2010/0212507 by Hester et al., and US application 2005/0118268 by Percel et al.

The following US patents have been incorporated by reference. U.S. Pat. No. 6,902,751 by Schleifenbaum et al., U.S. Pat. No. 5,958,502 y Fulger et al., U.S. Pat. No. 5,709,895 by Tanaka et al. U.S. Pat. No. 5,601,865 by Fulger et al., U.S. Pat. No. 5,399,368 by Garwood et al., U.S. Pat. No. 4,232,047 by Sair et al., U.S. Pat. No. 3,431,339 by Gyarmathy et al., U.S. Pat. No. 2,004,957 by Messner, U.S. Pat. No. 5,021,249 by Bunick et al., U.S. Pat. No. 4,689,235 by Barnes et al., U.S. Pat. No. 6,204,243 by Posanski, U.S. Pat. No. 5,401,512 by Rhodes et al., U.S. Pat. No. 6,663,888 by Percel et al., U.S. Pat. No. 6,500,454 by Percel et al., and U.S. Pat. No. 7,048,945 by Percel et al.

### BACKGROUND OF THE INVENTION

The preparation of a beverage with ingredients that retain their flavor over a long period of time or retain a relatively mixed or homogeneous composition through the drinking process can be complicated. Additionally, while it may be desirable to orally administer pharmaceutical compounds, vitamins, or other active compounds, including herbal ingredients, in a liquid beverage, the variety of ingredients found in these beverages and the potential effect they may have on the compound being administered has prevented their use for this purpose. This is further complicated by the fact that many beverages contain a variety of ingredients with different densities that may be hydrophobic or hydrophilic substances, further adding to the difficulty of maintaining a near homogeneous mix of beverage ingredients over a period of time.

For instance, the blending of a mixed drink such as coffee or tea requires the preparer to mix together a variety of different ingredients. There is a large number of coffee recipes, each requiring different ingredients, such as for example caffè Americano, café au lait, café Bombóm, café mélange, cafe mocha, Ca phe sua da, café cortado, eiskaffee, espresso Romano, flat white, Indian filter coffee, Kopi susu, caffè macchiato mazagran, mochasippi, Turkish coffee, Vienna coffee, yuanyang, etc.

Furthermore, there are a number of coffees based on liquors including but not limited to Irish coffee (Whiskey), Brandy coffee (Brandy), keoke coffee (Brandy and Kahl a), English coffee (Gin), calypso coffee (Tia Maria or Kahl a and Rum), Jamaican coffee (Tia Maria & Rum), shin shin coffee (Rum), Baileys Irish cream coffee, monk's coffee (Benedictine), Seville coffee (Cointreau), witch's coffee (Strega), Russian coffee (Vodka), priest coffee (Brennivin), Corfu coffee (Koum Quat liquor), kaffee fertig (coffee with Swiss prune schnapps), caffe corretto. Coffee is sold and used in mixed drinks in the form of beans, ground beans, or in instant-mix powders.

There are a large number of tea recipes including but not limited to white tea, yellow tea, green tea, oolong tea, black tea, pu'er tea, and a much larger number of herbal teas. Tea and coffee additives can include sweeteners such as honey, and flavors such as mint. Tea comes in leaves, in bags or in powder.

Many other beverages exist beside mixed drinks that include coffees and teas, which require their own ingredients and are sold in their own unique packages. These include but are not limited to beer, wine, sangria, cider, hot cider, hot chocolate, holicks, etc.

Sometimes the preparation of a multi-ingredient beverage requires that a base liquid be added such as for example water, milk, soda water, wine, cider, beer, ethyl alcohol, or fruit juice.

The large range of ingredients used in beverage preparation presents a problem should the beverage also contain ingredients designed for long-term flavor delivery, color maintenance, vitamin augmentation, or delivery of pharmaceutical or herbal compounds.

In addition to the complexity of beverage preparation, is the problem of loss of taste through the phenomenon of habituation, which manifests when a sense is exposed to a stimulus for a period of time and thereby it loses its sensitivity to that stimulus. More particularly, a drink's flavor that may appear to be intense initially loses some of its intensity after the taste buds become used to that flavor. The prior art does not adequately address this problem.

Additionally, many vitamins, pharmaceutical compounds, or other active compounds including herbal ingredients might be added to a beverage for the purpose of providing ingredients that should be processed in the stomach during digestion or otherwise broken down and absorbed through the intestine in order to become efficacious. However, the prior art does not provide a method for effective delivery of these ingredients, using an oral delivery method involving a liquid beverage, in a manner that protects the relevant ingredient so that it is processed and absorbed to maximize exposure to and effectiveness of a delivered compound.

Furthermore, there are certain combinations of pharmaceutical compounds, vitamins, and other active compounds, known to have a synergistic effect, where the use of one compound influences the efficacy of another. An example of this is the relationship between codeine and paracetamol. The combination of the aforementioned compounds is known to have an increased analgesic effect. In addition, certain pharmaceutical compounds have known side effects, which may be minimized if absorbed before or after another compound known to treat those particular side effects. For example, antibiotics such as penicillin and amoxicillin are known to cause diarrhea, nausea, vomiting, and stomach pain. Nausea, for example, can be treated with bismuth subsalicylate. If bismuth subsalicylate is digested prior to the metabolism of an antibiotic, there may be a reduction in the side effects experienced by the consumer. Prior art does not address the simultaneous consumption of compounds to alleviate side effects or increase efficacy by using an oral delivery method involving a liquid beverage.

In addition, many consumers are required to take multiple medications daily, and sometimes in a particular order. There is no prior art available addressing the use of an oral delivery method involving a liquid beverage to take multiple, different compounds per day in a particular order.

It is therefore one object of this invention to simplify the task of complex drink preparation. It is another object of the invention to mitigate the phenomenon of habituation and sustain a drink's taste intensity. Additionally, the invention provides for delayed-release drink color enhancement. The invention may also be used for the oral administration of pharmaceutical compounds, vitamins, or other active compounds including herbal ingredients to a person or veterinary patient in a liquid beverage.

Further features, aspects, and advantages of the present invention over the prior art will be more fully understood when considered in light of the following detailed description and claims.

As such, considering the foregoing, it may be appreciated that there continues to be a need for novel and improved devices and methods for delivering flavors and medications.

### SUMMARY OF THE INVENTION

The foregoing needs are met, to a great extent, by the present invention; wherein in aspects of this invention, enhancements are provided to the existing models of administering of flavors and medication to beverages, in the form of various embodiments of specially designed capsules and related devices.

The phenomenon of habituation is well known and well documented. It refers to a type of non-associative learning in which repeated exposure to a stimulus leads to decreased response to it. This phenomenon is responsible for after-images seen in the eyes after the retina has been exposed to an image. The same phenomenon applies to other senses, in particular, the sense of taste. Related to this, the second sip of a drink is usually less “tasty” or stimulating to the taste buds than the first. In an aspect, this invention mitigates the habituation phenomenon and sustains the intensity of a drink tasting experience by varying or modulating the taste of a beverage, over time. It utilizes time-release technologies involving capsules to expose the taste buds to flavors in a drink over an extended period, thereby exciting the sense of taste over a longer period of time.

Aspects of the present invention may be used for administration of delayed-release flavor or color enhancement of beverage preparations. It may also be used for the oral administration of pharmaceutical compounds, vitamins, or other active compounds, including herbal ingredients, to a person or animal in a liquid beverage.

In addition, aspects of this invention provide an added practical benefit in that it streamlines the task of the beverage preparer by combining, into a single capsule, a number of beverage ingredients that may be deposited into the liquid base for faster and easier preparation of mixed drinks.

In one aspect, the invention comprises a capsule filled with a matrix that contains (sequestered inside) the primary flavor, which suffuses through the beverage after the matrix has dispersed or dissolved in the beverage. Included inside this matrix are additional flavors captured in inner bodies that are designed to dissolve at later time intervals after they are released from the matrix. These inner bodies contain secondary, tertiary, and additional flavors that release after the primary beverage flavor response of the taste buds has subsided. In another beneficial aspect of the invention, a large number of inner bodies sequestered in a capsule produce a more complex flavor-time profile for the person drinking the beverage.

In this aspect, the invention comprises a capsule for delayed release of one or more flavor ingredients in a beverage, the timed releases occurring in multiple stages due to the gradual degradation or dissolution of the capsule constituents, which results in a delayed release of flavor trapped within the capsule. The capsule comprises:
a) A shell composed of material soluble in the aqueous beverage when a predetermined condition is met. Examples of such condition include a temperature level or shaking of the liquid. The dissolution of the shell starts the multi-stage capsule degradation process;
b) A liquid, solid, or powder matrix material soluble in the aqueous beverage. The dissolution of the matrix occurs in the second stage, after the shell has dissolved sufficiently to expose the matrix to the liquid;
c) Inner bodies inserted, embedded, or floating in the matrix. The inner bodies are configured to dissolve in the aqueous beverage in stages subsequent to the first and second stages, and after they are released from the matrix.

The shell, matrix, and inner bodies may optionally contain additional ingredients that are released into the beverage in corresponding stages. The invention includes embodiments wherein a variety of different types of inner bodies are assembled in coating layers, each layer having a predetermined rate of dissolution that is designed to achieve the desired time-release profile for the capsule's constituent ingredients. These additional inner bodies may include color enhancers that, when released into the beverage, change the hue of the beverage over time.

In another aspect, a composite timed release capsule can include:
a) A first layer, containing a first material that is water soluble;
b) A second layer, containing a second material that is water soluble, and which can further comprise inner bodies;
c) A third layer, also called an inner layer, which contains a third material.

In yet another aspect, a composite straw can include:
a) A standard straw;
b) A snap-off cap; which is connected to one end of the standard oversize straw and can be easily removed;
c) An upper stop, which allows passage of liquids;
d) A lower stop, which allows passage of liquids;
e) At least one timed release capsule that is positioned between the snap-off cap and the upper stop;
f) At least one composite timed release capsule, that is positioned between the upper stop and the lower stop;
whereby a user can remove the snap-off cap and pour the timed release capsules into a drink, and subsequently insert the composite straw into the drink such that the composite timed release capsule will gradually dissolve into the beverage, and allow the embedded inner bodies to fall through the lower stop into the drink.

In various aspects, the invention can also include capsule embodiments designed for oral administration of pharmaceutical compounds, vitamins, or other active compounds including herbal ingredients to a person or veterinary patient in a liquid beverage, according to a predetermined time profile. These compounds may be placed in inner bodies that are inserted, embedded, or floating in the matrix. Because the ingredients of these inner bodies are exposed to liquid and processed at a later time period, this timed release profile is advantageous for the delivery of compounds that should be processed in the stomach or digestive track to be efficacious. In addition, the order of which the compounds dissolve can be advantageous to increase the efficacy of the compound or compounds, or limit the potential harmful side effects of the previous or subsequent compounds. The invention could also be used to administer compounds to patients who take multiple pharmaceuticals per day and in a particular order.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. In addition, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a cross-sectional view of a timed release capsule, according to an embodiment of the invention.

FIG. 2 is a schematic diagram illustrating flavor intensity habituation curves, according to an embodiment of the invention.

FIG. 3 is a cross-sectional view of a layered timed release capsule, according to an embodiment of the invention.

FIG. 4 is a cross-sectional view of a composite timed release capsule, according to an embodiment of the invention.

FIG. 5 is a schematic view of a composite timed release capsule embedded in a standard straw, according to an embodiment of the invention.

FIG. 6 is a schematic view of a composite straw, according to an embodiment of the invention.

FIG. 7 is a schematic view of a composite straw, according to an embodiment of the invention.

FIG. 8 is a schematic view of a timed release drink container, according to an embodiment of the invention.

### DETAILED DESCRIPTION

Before describing the invention in detail, it should be observed that the present invention resides primarily in a novel and non-obvious combination of elements and process steps. So as not to obscure the disclosure with details that will readily be apparent to those skilled in the art, certain conventional elements and steps have been presented with lesser detail, while the drawings and specification describe in greater detail other elements and steps pertinent to understanding the invention.

The following embodiments are not intended to define limits as to the structure or method of the invention, but only to provide exemplary constructions. The embodiments are permissive rather than mandatory and illustrative rather than exhaustive.

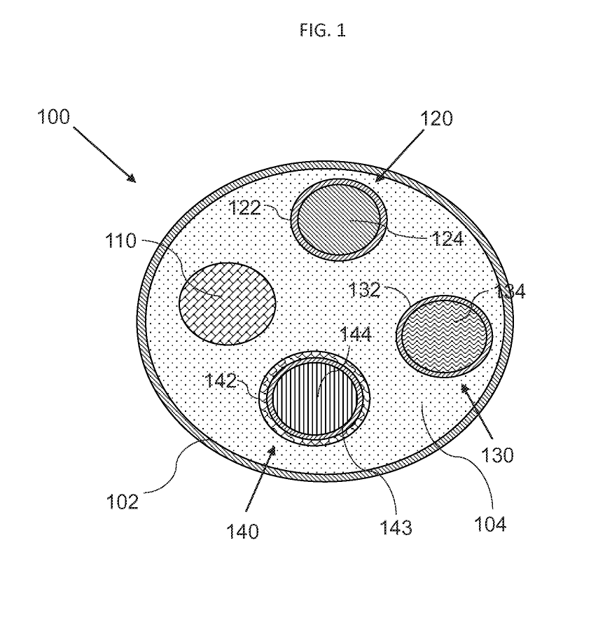

In the following, we describe the structure of an embodiment of a timed release capsule 100 with reference to FIG. 1, in such manner that like reference numerals refer to like components throughout; a convention that we shall employ for the remainder of this specification.

An embodiment of the present invention comprises a capsule that contains the ingredients required to prepare a blended beverage that includes at least one time-release component. When the capsule is dissolved in liquid, such as for example water, milk, alcohol or juice, flavors, colors, compounds and other constituents of the capsule can be released at various time intervals.

In an embodiment, illustrated in FIG. 1, a timed release capsule 100, can comprise:
a) A water-soluble capsule shell 102;
b) A matrix 104 material held by the capsule shell 102, such that the matrix 104 can be a solid, liquid, paste, gel, or powder;
c) At least one inner body 110 120 130 140, which is contained in the matrix 104, and can be embedded, suspended or floating in the matrix 104; and
d) At least one additional ingredient contained within inner bodies, which can include flavors, color, and pharmaceutical compounds, vitamins, or other active compounds including herbal ingredients.

In various related embodiments, the water-soluble capsule shell 102 can be made of a material that dissolves easily in water. Materials suitable for use as a time-release capsule shell are well known in the art, and can for example include hydroxyethyl cellulose.

In various related embodiments, the thickness of the capsule shell 102 can depend on its desired usefulness in a beverage delivery system and on how quickly the capsule is designed to dissolve in liquid preparations. The outside diameter of a timed release capsule 100 can typically be in a range of 1.5-3.5 inches and thickness of the capsule shell 102 can typically range from 1/16 to ⅛ of an inch. In some embodiments, outside diameter and shell thickness can, depending on application, fall outside these typical ranges.

In an embodiment, the composition of the capsule shell 102 can be selected to be soluble in liquid when a predetermined condition is met, such as certain time duration, a predetermined temperature, or a certain amount of agitation of the liquid. An inner body 110 120 130 140 typically ranges from ⅛ to ¾ of an inch in its outer diameter. These inner bodies are designed to fit well within a capsule and its constituent matrix. Optionally, these inner bodies are coated with a material that dissolves when exposed to liquid, such as hydroxyethyl cellulose.

For example, if a hot beverage is prepared, the capsule wall 102 can optionally begin to dissolve around 88° F. Alternatively, the capsule wall 102 may be designed to break open when the liquid it is contained within is vigorously shaken. In yet another embodiment of the present invention, the capsule wall can be relatively thick and only start to dissolve significantly after being exposed to liquid for at least 5 minutes.

The matrix 104 of FIG. 1 is composed of material that can easily dissolve or become a homogeneous mixture in liquid. It can provide the beverage with its initial flavor and color, and may typically comprise 25% to 75% sweetener ingredient and correspondingly, 75% to 25% flavor ingredient. For example, sugar and milk, sugar and mango, sugar and vanilla may be used in the capsule matrix as a flavored sweetener that is released into the beverage upon dissolution.

The matrix 104 of FIG. 1 holds at least one inner body 110 120 130 140, which is designed to hold additional flavors and colors. These inner bodies dissolve in the liquid as the matrix 104 releases them. Dissolving of the inner bodies 110 120 130 140 is the second stage of capsule dissolution in this timed-release process. The inner bodies' dissolution rate can be controlled or driven by the size and shape of the inner bodies, by the content and amount of matrix that holds them, and by the thickness and type of coatings on the capsule or on the inner bodies directly.

In a related embodiment, FIG. 1 shows various types of inner bodies, including:
a) A uniform inner body 110, which comprises a material of uniform density;
b) A coated inner body 120, wherein the coated inner body comprises an inner material 124, which is covered by a coating 122 designed to slow down dissolution of the coated inner body 120;
c) A double-coated inner body 140, wherein the coated inner body comprises an inner material 144, which is covered by an inner coating 143, which is further covered by an outer coating 144, wherein both coatings 143 144 are designed to slow down dissolution of the coated inner body 140; and
d) A vesicular type inner body 130 that contains a liquid 134 enclosed by a membrane 132.
A large number and variety of inner bodies may be incorporated within the capsule, according to various related embodiments of the present invention.

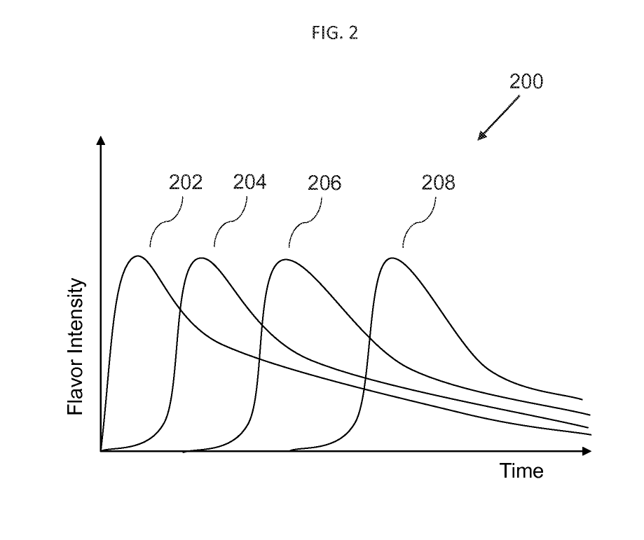

FIG. 2 illustrates how the phenomenon of habituation reduces the intensity of the flavor response over time. As a first flavor is presented to the palate, its intensity 202 peaks after the initial contact, and then decreases over time as the contact continues. A second flavor, followed by a third, and a fourth, are presented to the palate—all flavors following the same pattern of peaking and decaying intensity 204 206 208 according to the palate reaction. This invention intensifies the taste sense and flavor experienced from the drinking experience because the beverage is releasing flavors (whether consistent or different) over time. A larger number of inner bodies containing flavors mean that the beverage containing one or more capsules of the present invention yields a more complex time-flavor profile. Flavors carried by inner bodies can for example include mint, vanilla, anis, coffee, chocolate, salt, pepper, lemon oil, orange oil, walnut oil, nut oils, etc.

The capsule of FIG. 1 may also be used to deliver and release color in the beverage over time. Thus, a beverage may be prepared that changes color over time, as the color is released from the capsule. The color may optionally reside in the matrix 104 of FIG. 1 or the inner bodies 110 120 130 140. Color may also be mixed with flavor, liquid, or other compounds within coated or vesicular type inner bodies 120 130.

Additionally, the timed release capsule 100 may also be used to deliver and release pharmaceutical compounds, vitamins, or other active compounds, including herbal ingredients, to a person or animal through oral administration in a beverage, over time. Thus, a beverage may be prepared that releases compounds in the inner bodies during digestion in the stomach or in the intestine, after the capsule and matrix degrade and release the inner bodies. The present invention may be used for the oral administration of compounds to a person or animal in a liquid beverage.

### Example 1

In an example embodiment, a timed release capsule 100 can be designed to deliver a flavored coffee beverage by dissolving it in hot water, wherein the timed release capsule 100 further comprises:
a) A capsule shell 102, composed of the following ingredients by weight:

| | |
|---|---|
| Magnesium stearate | 37% |
| Hydroxyethyl cellulose | 12% |
| Glycerin | 11% |
| Gelatin | 22% |
| Stearic acid | 6% |
| Coloring | 4% |
| Water | 8% |
| Total | 100% |

b) A matrix 104, comprising by weight:

| | |
|---|---|
| Sugar | 40% |
| Milk | 36% |
| Vegetable oil | 10% |
| Vitamin A | 8% |
| Coloring (white/brown) | 4% |
| Water | 2% |
| Total | 100% |

c) Inner bodies, including:
   i. #1 inner body 120
      1. #1 inner body coating 122, comprising by weight:

| | |
|---|---|
| Magnesium stearate | 52% |
| Hydroxyethyl cellulose | 12% |
| Glycerin | 8% |
| Gelatin | 14% |
| Coloring | 6% |
| Water | 8% |
| Total | 100% |

2. #1 inner body material 124, comprising by weight:

| | |
|---|---|
| Natural Hazelnut, powder | 80% |
| Vitamin B12 | 8% |
| Coloring (Dark/Brown) | 2% |
| Water | 4% |
| Preservatives | 6% |
| Total | 100% |

ii. #2 Inner Body 120
   1. #2 inner body coating 122, comprising by weight:

| | |
|---|---|
| Magnesium stearate | 64% |
| Hydroxyethyl cellulose | 12% |
| Glycerin | 10% |
| Coloring | 6% |
| Water | 8% |
| Total | 100% |

2. #2 inner body material 124, comprising by weight:

| | |
|---|---|
| Artificial Hazelnut, powder | 80% |
| Nutrients & Vitamin B6 | 8% |
| Coloring (Dark/Brown) | 2% |
| Water | 4% |
| Preservatives | 6% |
| Total | 100% |

iii. #3 Inner Body
   1. #3 inner body coating 122, comprising by weight:

| | |
|---|---|
| Magnesium stearate | 44% |
| Hydroxyethyl cellulose | 20% |
| Glycerin | 6% |
| Gelatin | 16% |
| Coloring | 6% |
| Water | 8% |
| Total | 100% |

2. #3 inner body material 124, comprising by weight:

| | |
|---|---|
| Coffee, Instant Powder | 80% |
| Vitamin B2 | 8% |
| Coloring (Dark/Brown) | 2% |
| Water | 4% |
| Preservatives | 6% |
| Total | 100% |

### Example 2

In another example embodiment, a timed release capsule 100 can be designed to deliver a mango-flavored water, with high vitamin content, in an energy drink by dissolving the capsule in cold water, wherein the timed release capsule 100 further comprises:
a) A capsule shell 102, composed of the following ingredients by weight:

| | |
|---|---|
| Magnesium stearate | 37% |
| Hydroxyethyl cellulose | 12% |
| Glycerin | 11% |
| Gelatin | 22% |
| Stearic acid | 6% |
| Coloring | 4% |
| Water | 8% |
| Total | 100% |

b) A matrix 104, comprising by weight:

| | |
|---|---|
| Dextrose, Sugar | 40% |
| Guar Gum | 8% |
| Energy Blend (Taurine, | 46% |
| Glucuronolactone, Malic acid, | |
| N-acetyl, L-trysine, Guarana, | |
| Caffeine) | |
| Yellow coloring | 4% |
| Water | 2% |
| Total | 100% |

d) Inner bodies, including a
   i. #1 inner body, further comprising:
      1. #1 inner body coating 122, comprising by weight:

| | |
|---|---|
| Magnesium stearate | 52% |
| Hydroxyethyl cellulose | 12% |
| Glycerin | 6% |
| Gelatin | 14% |
| Nutrients | 6% |
| Coloring | 6% |
| Water | 4% |
| Total | 100% |

2. #1 inner body material 124, comprising by weight:

| | |
|---|---|
| Natural Mango, Powder | 80% |
| Vitamin B12 | 8% |
| Coloring (orange/yellow) | 2% |
| Water | 4% |
| Preservatives | 6% |
| Total | 100% |

ii. #2 inner body 120, further comprising:
   1. #2 inner body coating 122, comprising by weight:

| | |
|---|---|
| Magnesium stearate | 64% |
| Hydroxyethyl cellulose | 12% |
| Glycerin | 10% |
| Coloring | 6% |
| Water | 8% |
| Total | 100% |

2. #2 inner body material 124, comprising by weight:

| | |
|---|---|
| Artificial Mango, Powder | 80% |
| Vitamin B6 | 8% |
| Coloring (orange/yellow) | 2% |
| Water | 4% |
| Preservatives | 6% |
| Total | 100% |

iii. #3 inner body 120, further comprising:
   3. #3 inner body coating 122, comprising by weight:

| | |
|---|---|
| Magnesium stearate | 44% |
| Hydroxyethyl cellulose | 20% |
| Glycerin | 6% |
| Gelatin | 14% |
| Coloring | 8% |
| Water | 8% |
| Total | 100% |

i. #3 inner body material 124, comprising by weight:

| | |
|---|---|
| Artificial Pine Apple | 80% |
| Vitamin C | 8% |
| Coloring orange/yellow | 2% |
| Water | 4% |
| Preservatives | 6% |
| Total | 100% |

### Example 3

In yet another example embodiment, a timed release capsule 100 can be designed to deliver an alcoholic beverage containing vitamins and flavors, by dissolving it in an alcoholic liquid, such as vodka, wherein the timed release capsule 100 further comprises:

a) A capsule shell 102, composed of the following ingredients by weight:

| | |
|---|---|
| Magnesium stearate | 37% |
| Hydroxyethyl cellulose | 12% |
| Glycerin | 11% |
| Gelatin | 22% |
| Stearic acid | 6% |
| Coloring | 6% |
| Water | 6% |
| Total | 100% |

b) A matrix 104, comprising by weight:

| | |
|---|---|
| Lemon Juice | 10% |
| Lemon oil | 36% |
| Sodium Benzoate | 10% |
| Vitamin B-Complex | 19% |
| Vitamin B-12 | 19% |
| Coloring (yellow) | 4% |
| Water | 2% |
| Total | 100% |

c) Inner bodies, including a
   i. #1 inner body 120, further comprising:
      1. #1 inner body coating 122, comprising by weight:

| | |
|---|---|
| Magnesium stearate | 52% |
| Hydroxyethyl cellulose | 12% |
| Glycerin | 6% |
| Gelatin | 14% |
| Coloring | 8% |
| Water | 8% |
| Total | 100% |

2. #1 inner body material 124, comprising by weight:

| | |
|---|---|
| Salt | 80% |
| Coloring (white/red) | 4% |
| Water | 8% |
| Preservatives | 8% |
| Total | 100% |

ii. #2 inner body 120, further comprising:
   1. #2 inner body coating 122, comprising by weight:

| | |
|---|---|
| Magnesium stearate | 64% |
| Hydroxyethyl cellulose | 12% |
| Glycerin | 8% |
| Coloring | 8% |
| Water | 8% |
| Total | 100% |

2. #2 inner body material 124, comprising by weight:

| | |
|---|---|
| Peppers | 80% |
| Coloring (white/red) | 6% |
| Water | 8% |
| Preservatives | 6% |
| Total | 100% |

iii. #3 inner body 130, further comprising:
   1. #3 inner body membrane 132, comprising by weight:

| | |
|---|---|
| Magnesium stearate | 44% |
| Hydroxyethyl cellulose | 20% |
| Glycerin | 6% |
| Gelatin | 16% |
| Coloring | 6% |
| Water | 8% |
| Total | 100% |

2. #3 inner body liquid 134, comprising by weight:

| | |
|---|---|
| Vinegars | 80% |
| Coloring (white/red) | 6% |
| Water | 8% |
| Preservatives | 6% |
| Total | 100% |

In various related embodiments, even though the above narrative describes the timed release of flavors found in inner bodies in a drink, the same approach may also be used to deliver a timed release of nutrients, such as vitamins or herbs, compounds, or visual effects, such as colors. In one aspect of the invention, a change in color may be synchronized with the change in flavor to indicate, for example, the point of maximum flavor intensity for a particular flavor. These changes are driven by release of the inner bodies from the capsule matrix and degradation of any additional protective coating located directly around the inner bodies, which allows their ingredients to mix with the beverage.

In another embodiment of the present invention, the capsule contents, including at least some of the inner bodies, can provide a person or veterinary patient with an oral dosage or administration of pharmaceutical compounds, vitamins, other active compounds, or herbal ingredients in a liquid beverage.

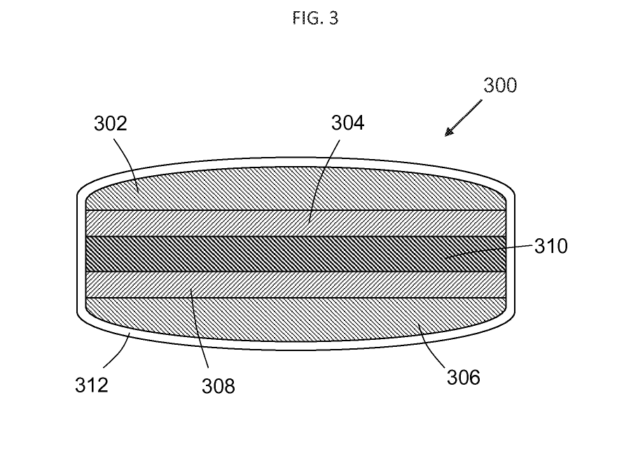

In another embodiment, as shown in FIG. 3, a layered timed release capsule 300, comprises different inner materials assembled in layers 302, 304, 306, 308, and 310, further covered with a coating 312. The coat dissolution rate is driven by the constituents and thickness of any capsule coating, the matrix, and any other optional layers of the capsule that affect the ingredient-release profile.

In a related embodiment, the capsule can incorporate pharmaceutical compounds, vitamins, herbal remedies, or other compounds used for a medical treatment. The thickness of the coating 312 and the layers 302 304 306 308 310 contribute to the ability to delay release of the compounds sequestered in the inner layers. The capsule size, coating and matrix thickness, and other properties can be adjusted to achieve delayed release profiles appropriate for oral delivery of compounds to children, adults, or veterinary patients, based on their digestive systems and digestive profiles. Such compounds delivered via oral administration in a capsule placed in a liquid beverage can include cold medicine such as Robitussin, digestive aids, such as Pepto-Bismol, or any other compound that is appropriate for oral ingestion and remains active when administered via the digestive system.

In yet another embodiment, a timed release capsule 100 can be specially formulated for veterinary patients. For example, when the patient is a cat, the capsule may include taurine or catnip to promote ingestion of the formulation.

In an additional embodiment, the timed release capsule 100 may be used to deliver three separate doses of pharmaceutical compounds, vitamins, other active compounds, or any combination of the aforementioned compounds. This embodiment could be used to deliver compounds in stages to minimalize potential side effects, increase the efficacy of the compounds or subsequent compounds, ensure the consumption of medication, or ensure the correct dosage of medication in the correct order.

In an example of minimizing potential side effects, amoxicillin can be used for the treatment of bacterial infections and is known to cause potential side effects like diarrhea, vomiting, or nausea. In contrast, bismuth subsalicylate can be used to treat diarrhea, heartburn, nausea, and upset stomach. In addition, vomiting and diarrhea can cause dehydration and electrolyte imbalance, which an aqueous solution of vitamins and electrolytes could replenish. Thus, the timed release capsule 100 may comprise of a matrix 104 of flavor, which dissolves in an aqueous solution; an inner body 110 of vitamins and electrolytes, delivered when a first condition is met; an inner body 120 containing bismuth subsalicylate as the inner material 124, delivered when a second condition is met generally after the first dose; and an inner body 130 containing a liquid dosage of amoxicillin as the liquid 134, which is delivered when a third condition is met and generally after the first and second dose is delivered.

In an example of increasing the efficacy of a compound, published reports have shown that certain drugs have a low absorption at high gastric pH levels. In a related embodiment, the timed release capsule 100 may comprise of a matrix 104 of a certain flavor, which dissolves in an aqueous solution when a first condition is met; an inner body 120 comprised of a compound 124 to lower the gastric pH levels, which dissolves when a second certain condition is met; and an inner body 130 containing a compound that absorbs affectively at lower gastric pH levels, which is delivered when a third condition is met.

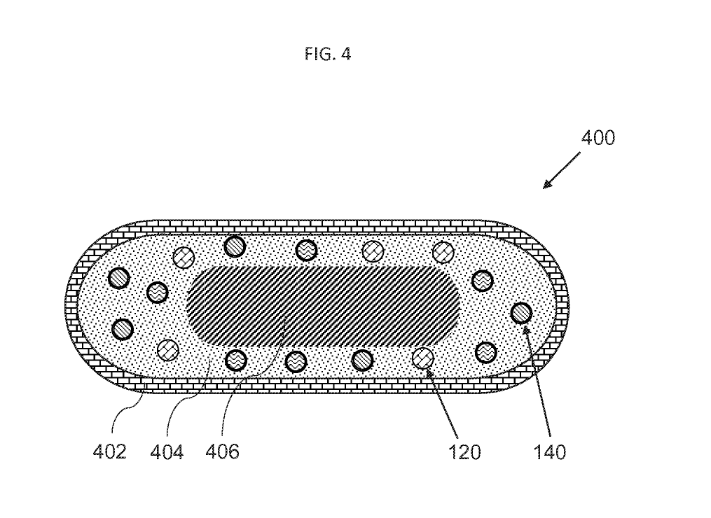

In an embodiment, as illustrated in FIG. 4, a composite timed release capsule 400 can comprise a plurality of nested layers, such that an inner layer further contains embedded inner bodies.

In a related embodiment, a composite timed release capsule 400, can comprise:
a) A first layer 402, comprising a first material, such that the first material is water soluble;
b) A second layer 404, comprising a second material, such that the second material is water soluble; wherein the second material can further comprise inner bodies 110 120 130 140; and
c) A third layer 406 also referred to as an inner layer 406, comprising a third material, such that the second material is water soluble.

In a further related example embodiment, the composite timed release capsule 400, can be structured, such that:
a) the first material comprises a flavoring and/or coloring agent;
b) the second material comprises a first pharmaceutical agent, which is known to cause stomach irritation as a side effect;
c) the inner bodies are comprised of:
   i. coated inner bodies 120, wherein the coating 122 is configured to not break down in an aqueous solution, and further configured to dissolve in an acidic environment, wherein further a first inner material 124 comprises a second pharmaceutical agent, which is known to reduce stomach irritation, such as for example an antacid designed for delivery in the stomach; and
   ii. double-coated inner bodies 140, wherein the outer coating 144 is configured to not break down in an aqueous solution, and the outer coating is further configured to dissolve in an acidic environment, and wherein further the inner coating 143 is an enteric coating, which will not dissolve in acidic environment, but will dissolve in a non-acidic aqueous solution, wherein further the second inner material 144 comprises a third pharmaceutical agent for delivery in the small intestines of a human or a pet, such as for example pain medication for absorption in the small intestines to minimize risk of stomach irritation or inflammation;
d) the third material includes a water soluble coloring agent;
whereby the first, second, and third material will dissolve when the composite timed release capsule 400 is submerged in water, and dissolution of the third material will cause a color shift, which indicates the capsule is fully dissolved, such that the inner bodies are suspended in the water solution, without breaking down due to the outer coating, and whereby further both the coated inner bodies 120 and the double-coated inner bodies 140 will not dissolve in the beverage, such that the inner bodies 120 will dissolve in the stomach, and the double-coated inner bodies 140 will dissolve in the small intestines.

In a further related embodiment, the outer coating 144 can be any of a plurality of well-known coatings and pharmaceutical films, which will resist dissolution in water, but will dissolve rapidly in an acidic environment, such as the stomach of a human or a pet. Similarly, the inner coating 143 can be selected from well-known enteric coatings, such as for example a pharmaceutical glaze.

In various further related embodiments, the inner bodies 110 120 130 140 can have various densities, such that the inner bodies 110 120 130 140 can either become suspended or floating in an aqueous liquid, or can sink or float.

In a related embodiment, a change in color can let a user know that a drink is ready. In a further related embodiment, dissolution of the third material can cause a color shift of the drink, which can indicate that the drink is ready.

In a related embodiment, a beverage can undergo two or more color shifts during dissolution of a timed release capsule 400.

In a related embodiment, a beverage can become effervescent during the dissolution of a timed release capsule 100 or a composite timed release capsule 400. In a further related embodiment, a timed release capsule 100 can be configured such that the matrix 104 contains an effervescent agent, or a composite timed release capsule can be configured such that the third material contains an effervescent agent. The effervescent agent can for example be a mixture of citric acid and sodium bicarbonate. In a related embodiment, the matrix 104 can be pressure formed, such that no further coating 102 is necessary.

In a related embodiment, a timed release capsule 400 can be so designed as to permit use for more than one personal beverage, similar to reuse of a tea bag for brewing of more than one cup of tea.

In various related embodiment, a timed release capsule 400 can be configured in a plurality of different sizes. For example, a larger size a timed release capsule 400 can be configured for use with more than one personal beverage.

In various related embodiments, a timed release capsule 400 can further comprise:
a) Prebiotic materials, in the form of non-digestible food ingredients that stimulate the growth and/or activity of bacteria and/or other micro-organisms in the digestive system; and
b) Probiotic materials, in the form bacteria and/or other micro-organisms may have benefits for the digestive system.

In various further related embodiments, any layer 402 404 406 or inner bodies 110 120 130 140 of the timed release capsule 400 may comprise prebiotic and/or probiotic materials. In a yet further related embodiment, a timed release capsule 400 comprising both prebiotic and probiotic materials may provide a symbiotic function in the digestive system.

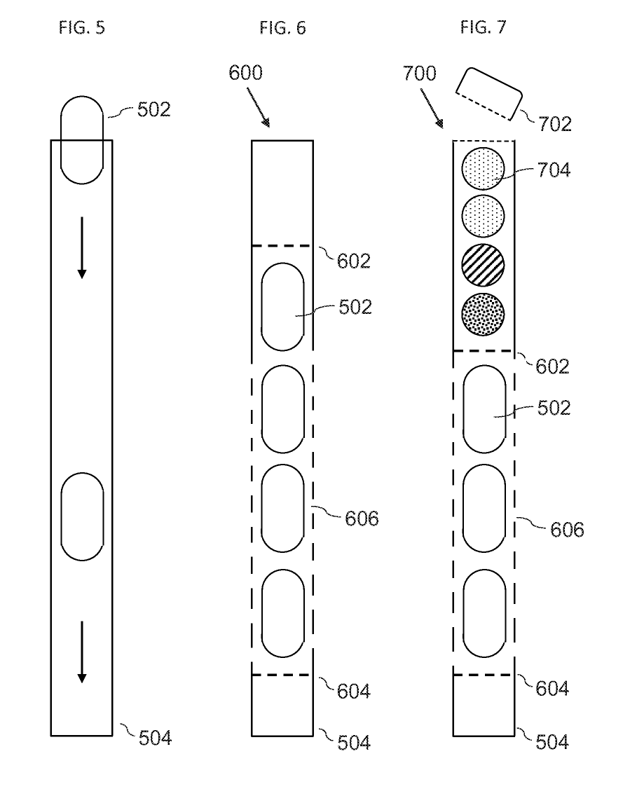

In a related embodiment, as shown in FIG. 5, the composite timed release capsule 400 can be configured with an elongated tubular shape, such that its diameter is in a range of 0.2-0.5 inches, such that the composite timed release capsule can fit inside a standard straw 504, of the oversized type commonly used for “Bubble Tea, tea based beverages with embedded tapioca pearls that fit inside the standard straw 504. This can allow a user to inserts the composite timed release capsule into a drink container through the standard straw 504, which can for example be convenient if the standard straw 504 is pierced through a plastic film covering the top of drink container.

In various other embodiments, the composite timed release capsule 400 can be configured with a variety of diameters, to fit inside a variety of standard straws 504, which can include thin, medium, standard, or oversize straws.

In an embodiment, a composite straw 600, as illustrated in FIG. 6, can be comprised of:
a) A standard straw 504;
b) An upper stop 602, wherein the upper stop 602 is perforated to allow passage of liquids;
c) A lower stop 604, wherein the lower stop 604 is perforated to allow passage of liquids;
d) At least one composite timed release capsule 400, such that the composite timed release capsule 400 is retained in position between the upper stop 602 and the lower stop 604;
whereby a user can insert the composite straw 600 into a drinking container filled with a beverage, such the composite timed release capsule 400 will gradually dissolve into the beverage, such that the inner bodies 120 140 are released.

In an embodiment, a composite straw 700, as illustrated in FIG. 7, can be comprised of:
a) A standard straw 504;
b) A snap-off cap 702; which is configured with a water and moisture tight connection to the standard straw 504; such that the connection is adapted, for example with a partial perforation, to allow easy removal;
c) A upper stop 602, wherein the upper stop 602 is perforated to allow passage of liquids;
d) A lower stop 604, wherein the lower stop 604 is perforated to allow passage of liquids;
e) At least one timed release capsule 100; such that the timed release capsule 100 is retained in position between the snap-off cap 702 and the upper stop 602;
f) At least one composite timed release capsule 400, such that the composite timed release capsule 400 is retained in position between the upper stop 602 and the lower stop 604;
whereby a user can remove the snap-off cap in order to pour the timed release capsules into a drinking container which contains a beverage, and subsequently insert the composite straw 600 into the drinking container such that the composite timed release capsule 400 will gradually dissolve into the beverage, such that the inner bodies 120 140 are released.

In various embodiments, the timed release capsules 100 and/or composite timed release capsules 400 can be packaged with beverages or beverage containers such that the timed release capsules 400 can be released into the beverage before consumption.

In a related embodiment, the inner bodies 120 140 can have a density greater than 1 gram/cm³, such that the released inner bodies can fall through the lower stop into the beverage.

In a related embodiment, the inner bodies 120 140 can have a density substantially similar to 1 gram/cm³, such that the released inner bodies can become suspended in the beverage inside the straw, and the user can subsequently ingest the inner bodies directly from the composite straw 600 700.

In a related embodiment, the wall of the straw 504, can further include perforations 606, to allow for passage of dissolved parts of the timed release capsules 400 to pass into the full volume of the beverage.

In a related embodiment, the wall of the straw 504, can further include perforations 606, to allow for passage of dissolved parts of the timed release capsules 400 to pass into the full volume of the beverage.

In an embodiment, a composite straw 600 or a composite straw 700 can include a first composite timed release capsule 400, which is designed to control the flow of a beverage, for example to prevent flow of a hot beverage, such as near boiling coffee or tea. In a related embodiment, the first composite timed release capsule 400 can include a coating which has retrograde or inverse temperature dependent solubility, such that the coating is almost insoluble above a pre-determined temperature, which typically can be in a range from 68 degrees Celsius to 100 degrees Celsius. In a further related embodiment, the first material of the first composite timed release capsule 400 can have retrograde or inverse temperature dependent solubility. In a further related embodiment, the first composite timed release capsule 400, can be positioned above other timed release capsules 400, and above any perforations 606 of the straw 504 wall.

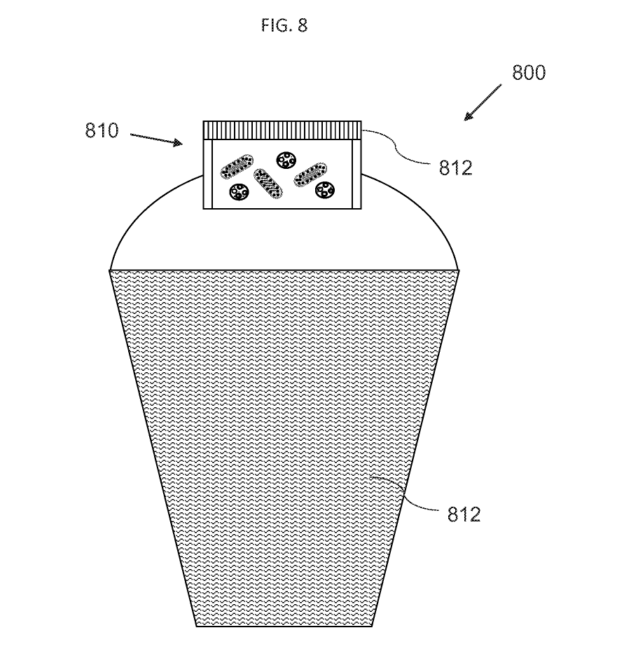

In a related example, illustrated in FIG. 8, a timed release drink container 800 can comprise timed release capsules 100 and/or composite timed release capsules 400, which can be stored in a capsule compartment 810, which can further comprise a screw lid 812, which can allow release of the capsules into a beverage 812.

One advantage of various embodiments of this invention is that they can resolve the problem of incompatible formulas, in which formula components would not be stable or form homogeneous solutions if mixed together at manufacturing time, and therefore give the manufacturer a wide range of new options concerning the various components that may be used. In other words, a capsule can be manufactured at one time and consumed at another time. Its components such as the shell, the matrix, or any one of the inner bodies could comprise chemicals or other compounds that lose some or all of their activity if they were combined or come in contact with each other during the manufacturing process. The solution provided by this invention involves the mixing of these constituents at the time of dissolution in a beverage, thereby avoiding or reducing concerns regarding loss of activity.

The many features and advantages of the invention are apparent from the detailed specification, and thus, it is intended by the appended claims to cover all such features and advantages of the invention, which fall within the true spirit and scope of the invention.

Many such alternative configurations are readily apparent, and should be considered fully included in this specification and the claims appended hereto. Accordingly, since numerous modifications and variations will readily occur to those skilled in the art, it is not desired to limit the invention to the exact construction and operation illustrated and described, and thus, all suitable modifications and equivalents may be resorted to, falling within the scope of the invention.

What is claimed is:

1. A composite timed release capsule, comprising:
a) a first layer, comprising a first material, such that the first material is water soluble;
b) a second layer, comprising a second material inside of the first material, such that the second material is water soluble; wherein the second material further comprises inner bodies;
whereby the first and second materials will dissolve when the composite timed release capsule is submerged in an aqueous solution.

2. The composite timed release capsule of claim 1, wherein the inner bodies further comprise:
at least one coated inner body, further comprising a coating which is configured to not break down in an aqueous solution, and further configured to dissolve in an acidic environment.

3. The composite timed release capsule of claim 1, wherein the inner bodies further comprise:
at least one double-coated inner body, wherein an outer coating is configured to not break down in an aqueous solution, such that the outer coating is further configured to dissolve in an acidic environment, and wherein further an inner coating is an enteric coating which will not dissolve in acidic environment, but will dissolve in a non-acidic aqueous solution.

4. The composite timed release capsule of claim 1, further comprising:
c) a third layer, comprising a third material, which is water soluble, such that the third material is inside the second material.

5. The composite timed release capsule of claim 1, wherein the first material further comprises a flavoring agent.

6. The composite timed release capsule of claim 1, wherein the first material further comprises a coloring agent.

7. The composite timed release capsule of claim 4, wherein:
a) the first material, is further configured with a first predetermined condition of solubility;
b) the second material, is further configured with a second predetermined condition of solubility;
c) the third material, is further configured with a third predetermined condition of solubility.

8. The composite timed release capsule of claim 7, wherein the first condition of solubility is a temperature of the aqueous solution.

9. The composite timed release capsule of claim 7, wherein the first condition of solubility is a time duration of exposure to the aqueous solution.

10. The composite timed release capsule of claim 7, wherein the first condition of solubility is an amount of shaking of the aqueous solution.

11. The composite timed release capsule of claim 4, wherein the third material further comprises a coloring agent, whereby dissolution of the coloring agent into the aqueous solution causes a color shift of the aqueous solution that indicates the aqueous solution is ready for drinking.

12. The composite timed release capsule of claim 1, wherein the composite timed release capsule is chewable.

13. The composite timed release capsule of claim 1, wherein the composite timed release capsule is edible.

14. The composite timed release capsule of claim 1, wherein the first material further comprises a food material selected from the group consisting of mint, vanilla, anis, coffee, chocolate, salt, pepper, lemon oil, orange oil, walnut oil and nut oils.

15. The composite timed release capsule of claim 1, wherein the second material further comprises a first pharmaceutical agent, which is known to cause stomach irritation as a side effect; and wherein further the inner bodies comprise:
a) coated inner bodies, further comprising a coating which is configured to not break down in an aqueous solution, and further configured to dissolve in an acidic environment, and further comprising a first inner material, comprising a second pharmaceutical agent, which is known to reduce stomach irritation;
b) double-coated inner bodies, further comprising:
an outer coating that is configured to not break down in an aqueous solution, such that the outer coating is further configured to dissolve in an acidic environment;
an inner coating that is an enteric coating, which will not dissolve in an acidic environment, but will dissolve in a non-acidic aqueous solution; and
a second inner material, comprising a third pharmaceutical agent for delivery in the small intestines of a human or a pet, whereby risk of stomach irritation or inflammation is reduced.

16. The composite timed release capsule of claim 15, wherein the third pharmaceutical agent is a pain medication.

17. The composite timed release capsule of claim 1, wherein the composite timed release capsule is configured with a diameter in a range of 0.2-0.5 inches;
whereby the composite timed release capsule fits inside a standard straw, whereby further the composite time released capsule allows a user to insert the composite timed release capsule into a drink container through the standard straw.

18. A composite straw, comprising:
a) a standard straw;
b) an upper stop, wherein the upper stop is perforated to allow passage of liquids;
c) a lower stop, wherein the lower stop is perforated to allow passage of liquids;
d) At least one composite timed release capsule, such that the composite timed release capsule is retained in position between the upper stop and the lower stop;
whereby the composite straw allows a user to insert the composite straw into a drinking container filled with a beverage, such the composite timed release capsule gradually dissolves into the beverage, such that inner bodies are released.

19. The composite straw of claim 18, further comprising
a) a snap-off cap, which is configured with a water and moisture tight connection to the standard straw, such that the connection is adapted to allow easy removal;
b) At least one timed release capsule, such that the timed release capsule is retained in position between the snap-off cap and the upper stop;
whereby the composite straw allows a user to remove the snap-off cap in order to pour the timed release capsules into a drinking container, which contains a beverage, and subsequently insert the composite straw into the drinking container such that the composite timed release capsule will gradually dissolve into the beverage, whereby the inner bodies are released.

20. The composite straw of claim 18, wherein the composite timed release capsule, further comprises:
a) a first layer, comprising a first material, such that the first material is water soluble;
b) a second layer, comprising a second material inside of the first material, such that the second material is water dissolvable; wherein the second material further comprises inner bodies;
whereby the first and second materials will dissolve when the composite timed release capsule is submerged in an aqueous solution.

21. The composite straw of claim 20, wherein the inner bodies further comprise:
at least one coated inner body, further comprising a coating which is configured to not break down in an aqueous solution, and further configured to dissolve in an acidic environment.

22. The composite straw of claim 20, wherein the inner bodies further comprise:
at least one double-coated inner body, wherein an outer coating is configured to not break down in an aqueous solution, such that the outer coating is further configured to dissolve in an acidic environment, and wherein further an inner coating is an enteric coating which will not dissolve in acidic environment, but will dissolve in a non-acidic aqueous solution.

23. The composite straw of claim 20, wherein the composite timed release capsule, further comprises a third layer, comprising a third material, such that the third material is inside the second material.

24. The composite straw of claim 20, wherein the second material further comprises a first pharmaceutical agent, which is known to cause stomach irritation as a side effect; and wherein the inner bodies further comprise:
a) coated inner bodies, further comprising a coating which is configured to not break down in an aqueous solution, and further configured to dissolve in an acidic environment, and further comprising a first inner material, comprising a second pharmaceutical agent, which is known to reduce stomach irritation;
b) double-coated inner bodies, further comprising:
an outer coating that is configured to not break down in an aqueous solution, such that the outer coating is further configured to dissolve in an acidic environment;
an inner coating that is an enteric coating, which will not dissolve in an acidic environment, but will dissolve in a non-acidic aqueous solution; and
a second inner material, comprising a third pharmaceutical agent for delivery in the small intestines of a human or a pet, whereby risk of stomach irritation or inflammation is reduced.

25. The composite straw of claim 20, wherein the third pharmaceutical agent is a pain medication.

26. The composite straw of claim 20, wherein the timed release capsule further comprises:
a) a water-soluble capsule shell;
b) a matrix material held within the capsule shell.

27. The composite straw of claim 20, wherein the matrix further comprises at least one inner body, which is contained in the matrix.
